(19) United States
(12) Patent Application Publication
(10) Pub. No.: US 2003/0152979 A1
(43) Pub. Date: Aug. 14, 2003

(54) Methods for reducing contamination by genetically modified plant products

(76) Inventor: Marko Stojanovic, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/320,040

(22) Filed: Dec. 16, 2002

Related U.S. Application Data
(60) Provisional application No. 60/353,839, filed on Jan. 31, 2002.

(51) Int. Cl. C12Q 1/68

(52) U.S. Cl. 435/6

(57) ABSTRACT

Disclosed is a method for determining the relative number of genetically modified plant products in a population of plants so as to characterize the plant products as being either “genetically modified” or “non-genetically modified,” the method comprising genetic testing of samples obtained from a representative number of the plants of the population.

Also disclosed is a method for reducing the level of contamination of non-genetically modified plant products with genetically modified plant products, the method comprising the steps of: (a) utilizing data obtained from genetic testing of samples obtained from plants from a plurality of populations of plants prior to combining plant products from the different populations; and (b) determining whether plants from any of the populations are genetically modified.

### CROSS REFERENCE TO RELATED APPLICATIONS

This application claims priority to U.S. Provisional Application No. 60/353,839, filed Jan. 31, 2002.

### FIELD OF THE INVENTION

The present invention relates to a method for reducing the level of contamination of non-genetically modified plant products with genetically modified plant products.

### BACKGROUND OF THE INVENTION

Genetically modified (GM) plant products are increasingly being introduced into the world's food supply. This introduction is of high interest to many consumers and various regulatory agencies around the world are very interested in establishing whether genetically engineered components are present in edible products entering their countries. Buyers and sellers at each step in the supply chain, from raw material product to finished product, therefore need to know whether a given shipment of product meets applicable GM standards. Additionally, the question of whether a particular genetically engineered event has been approved for import into a country is an issue of considerable significance.

Significant work has been done in the area of designing processes for certifying the GM content/contamination for finished products. Some of these processes have utilized genetic testing in the certification process. For example, the prior art includes certification methods that involve genetic testing at the seed source, testing after plant products have been harvested and combined for shipping for further processing, and testing at various times after plant products have been combined by the entity that will prepare finished product from the plant product raw materials.

While genetic testing of the seed source is certainly beneficial, as is the testing of the field where growing is to take place, there are circumstances where plants grown from GM-free seed sources in GM-free fields can become contaminated. For example, contamination can occur as a result of crosspollination from plants of an adjacent field that contains GM product, or due to “volunteers” remaining from the previous year's planting. Similarly, while there are clear benefits with genetic testing of plant products from one field or source (referred to herein as a “population”) after those products have been combined with plant products from another field or source, it is possible that at this stage GM and non-GM plant products have been combined and the entire combined lot must be characterized and subsequently processed as “GM containing” product.

Accordingly, there is a need for a method that tests plant products in a manner such that GM-free plant products are not subsequently contaminated because of their combination with GM-containing plant products.

### SUMMARY OF THE INVENTION

In one aspect, the invention is directed to a method for determining the relative number of genetically modified plant products in a population of plants so as to characterize the plant products as being either “genetically modified” or “non-genetically modified,” the method comprising genetic testing of samples obtained from a representative number of the plants of the population.

In another aspect, the invention is directed to a method for reducing the level of contamination of non-genetically modified plant products with genetically modified plant products, the method comprising the steps of:

a. utilizing data obtained from genetic testing of samples obtained from plants from a plurality of populations of plants prior to combining plant products from the different populations; and

b. determining whether plants from any of the populations are genetically modified.

In this second aspect, the method will typically further comprise the step of combining plant products from only those populations that consist of non-genetically modified plants.

### DETAILED DESCRIPTION OF THE INVENTION

I. Definitions

The term “confidence level” means the probability that the value of a parameter falls within a specified range of values.

The term “comprising” means various components and processing steps can be conjointly employed in practicing the present invention. Accordingly, the term “comprising” encompasses the more restrictive terms “consisting essentially of” and “consisting of.”

The terms “farinaceous food” and “farinaceous food product” are used interchangeably and refer to products made with starches, such as fabricated crisps, tortillas, wheat thins, crackers (creamed and sandwiches), soft tortillas, rice cakes, cereals, extruded snacks, granola bars, Newton's, etc. Said farinaceous food products may be sweet, salty, or savory.

The term “finished product,” when used in the context of food products, refers to a consumable product that has been finished (e.g., baked, fried, frozen, dehydrated, freeze-dried, etc.) to produce a ready-to-eat product or requires modest additional processing to provide a consumable product (e.g., reconstitution of dehydrated potatoes in water)

The terms “genetically modified” (“GM”) and “non-genetically modified” (“non-GM”) are described below. As will be discussed, while the terms themselves are qualitative descriptions, that qualitative description may be based on statistical data that is either qualitative or quantitative.

The term “plant” means a living organism of the kingdom Plantae, usually containing chlorophyll enabling it to live wholly on inorganic substances and lacking specialized sense organs and the power of voluntary movement.

The term “plant product” means any product originated from a plant that ultimately enters the food chain.

The term “plant seed” means fertilized ovules of a plant or plants, especially in the form of small roundish bodies or grains dispersed naturally or collected for sowing to produce a new generation of plants.

The terms “population” and “population of plants” are used interchangeably and each means any finite or infinite collection of items under consideration.

II. The Present Methods

As brief background, detection of genetically modified materials currently falls into two general testing methodologies, both of which are well known in the art. One method, enzyme-linked immunosorbent assay (ELISA), detects the presence of the unique protein that is the product of the information encoded by the modified DNA in the genetically modified plant. Alternately, the polymerase chain reaction (PCR) method detects the actual DNA sequences that have been inserted into the modified plant's naturally occurring DNA.

In an ELISA test for genetically modified plant products, a kit manufacturer isolates the unique protein (resulting from the genetic modification) and raises antibodies against specific epitopes of this protein. In an assay, the proteins, if present, are bound by labeled antibodies. The presence of a genetically modified sample is indicated by a color reaction catalyzed by an enzyme linked to the antibodies.

PCR uses a primer (a portion of DNA) that targets a nucleotide sequence unique to the plant product in question. In this manner, PCR is used to determine if that GM plant product is present in the sample tested. More recently, PCR has been applied to two common scenarios. The first concerns whether a sample contains a genetically engineered component. In this test, the specific nature of the modification is not of particular importance. The second approach assesses whether a specific GM event is present in the sample. (To determine if a specific GM event is present in a sample, the testing facility will look for a nucleotide sequence that is unique to the GM event in question.) The response to both of these questions can be either a qualitative (i.e., positive-negative answer) or a quantitative determination of the relative amount of target DNA. In a qualitative system, the PCR is performed for a sufficient number of cycles to get maximum formation of replicated molecules.

PCR quantification is done by determining the ratio of target GM DNA to the total of the species DNA. This can be done using fluorescence-coupled real-time PCR machines, which include a thermocycler incorporated with an optical system. (E.g., Applied Biosystem ABI PRISM™ 7700 detector instrument (TaqMan).) The instrument detects laser-activated fluorescence signals in a common 96 well plate. In each test solution, an additional oligonucleotide labeled with a fluorescent dye is added (e.g., a TaqMan probe). This probe anneals, like an ordinary PCR primer, to a specific region of the single-stranded, unzipped DNA molecule. During the polymerization step, as the DNA polymerase builds the complementary strand, it encounters the annealed dye-labeled probe, hydrolyzes the probe, which causes a fluorescence emission. Fiber optic sensors over each well measure the change in fluorescence light intensity with the increasing number of cycles. Calibration curves are calculated from standards being run simultaneously and the percentage of target DNA versus total species DNA can be calculated.

The skilled artisan will recognize that various commercial laboratories are capable of performing either ELISA or PCR-based genetic testing. One such laboratory is GeneScan USA Inc. (Belle Chasse, La.).

In one aspect, the present invention is directed to a method for determining the relative number of genetically modified plant products in a population of plants so as to characterize the plant products of the population as being either ‘genetically modified’ or ‘non-genetically modified’, the method comprising genetic testing of samples obtained from a representative number of the plants of the population. It will be recognized that testing may occur either during the growing phase of the plant products or after products have been harvested but before products from one population have been combined with plant products of another population. It will be further recognized that in many instances, it will be preferred that testing (including genetic testing if desirable) can be performed in addition to the testing performed in accordance with the present invention. For example, it may be desired to conduct genetic testing on the seeds to be used for planting. Additionally, while the present invention seeks to prevent the combining of non-GM with GM-containing plant products, it may be necessary to conduct additional testing (e.g., following processing of the plant products into other finished products, such as farinaceous snacks) to ensure that contamination has not occurred later in the production chain. As such, the methods of the present invention may be a part of an overall process for establishing what is termed in the art as an Identity Preservation (IP) system. See U.S. Patent Application Publication No. 2001/0011437 by Shortridge et al. A representative system is summarized here.

Samples obtained from a population of plants are tested genetically and are deemed to be non-GM products according to a protocol established for that product. For example, with reference to the Example set forth below, all the samples come back negative indicating that there was not a single GM plant out of 4603 plants samples across the 3 fields that make up the population. In that example, the results indicate that the level of GM contamination in that population is not more than 0.067% at 95% confidence or not more than 0.1% at 99% confidence. Therefore, the potatoes harvested from the plants tested are certified for Japanese production of farinaceous foods. In order to use them for that production, IP procedures must be followed from the field until the actual consumption of dehydrated potatoes (e.g., potato flakes, potato flanules or potato granules) occurs. A typical IP procedure consists of the following steps (but is not limited by those or by the order presented below) that insure raw material (potato) tracking from seeds to finished product.

1. Ensure varietal purity of seed. Obtain seed from reputable seed companies (preferably companies that the supplier has a long term relationship).

2. During seeding of the fields ensure that the seeding equipment is cleaned and inspected between each seed source (that's also a standard procedure for industrial farmers).

3. During harvest, equipment needs to be cleaned and inspected between the fields.

4. Trucks used for transportation of potatoes from the field(s) to the storage sheds need to be inspected and cleaned during each round trip.

5. Potatoes in sheds should be grouped according to their GM content, and fields/populations (which may be determined by seed supply source) should be physically separated in sheds.

6. If potatoes are sent to a sorting station, step 4 needs to be repeated, and sorting station should ensure no cross contamination with any other product.

7. When bringing potatoes into the dehydration plant, clean and inspect receiving bins, transfer lines, cookers, drums, storage facilities, to prevent cross-contamination with any other product. Production does not necessarily need to stop, but in that case a long transition period is required (even then the receiving bins and transfer lines need to be empty and inspected). The transition time is at minimum 120 min. (35 min. in the cooker, 30 min. for the drum, and a safety margin of at least 55 min.).

8. Preferred method is direct loading into railcar/silo trucks/supersacks or any other transportation container/package, but if that is not feasible, a non-GM storage facility must be designated in the plant. Initially the non-GM storage facility must be completely emptied, cleaned and inspected. If that storage facility inadvertently becomes contaminated, the whole procedure must be repeated (empty/clean/inspect).

Each step of the IP procedure typically will be documented by the supplier and the documents are made available for auditing/traceability purposes.

In those instances where the plants in question do not require pollination to reproduce (e.g., potatoes (when cultivated as a crop)) or the plant product is other than the plant seed, it will be possible to take test samples from any portion of the growing plant, including but not limited to the plant stem, plant leaves, the plant flower, the plant root, the plant product itself, or any combination thereof. In those instances where the plants do require pollination to reproduce, to ensure the accuracy of results, it will be preferred to use samples from the plant product itself. This is to ensure that contamination has not occurred during the pollination of the plant in question.

As will be appreciated, the methods of the present invention are useful in ensuring the lack of contamination in a variety of plant types. For example, plants to be tested include but are not limited to fruits, vegetables, grains, tubers, cereals, and legumes.

With respect to the analysis conducted to establish whether plant products from a given population are “non-GM” or “GM”, the skilled artisan will recognize that the level of statistical rigor employed in making that determination is within the skilled artisan's discretion. Thus, one practicing the present methods will necessarily establish their own thresholds (e.g., in terms of relative levels of GM products in a population, confidence levels used) for determining whether a population is “non-GM”. In other words, while statistical analysis is obviously an aspect of the methods of the present invention, the methods aren't limited by the nature of those analyses. Rather, once a statistical definition for GM and non-GM is established for a given situation, the present methods can be employed to avoid contamination of non-GM with GM plant products.

The skilled artisan will also recognize that populations may be established based on various parameters. One preferred parameter is to segregate populations based on the seed origin (e.g., seed supplier).

While the present invention is not to be limited in terms of how the user establishes the threshold for the GM/non-GM characterization, the following is a discussion of representative statistical analyses.

| | Confidence level 95%, | Confidence level 99%, |
|---|---|---|
| Upper bound for fraction GM* | (No. of Samples - No GM | (No. of Samples - No GM plants |
| (% of total population) | plants detected**) | detected**) |
| 10 | 29 | 44 |
| 1 | 299 | 459 |
| 0.1 | 2,995 | 4,603 |
| 0.05 | 5,990 | 9,209 |
| | Confidence level 95%, | Confidence level 99%, |
| Upper bound for fraction GM* | (No. of Samples - 1 GM | (No. of Samples - 1 GM plant |
| (% of total population) | plant detected**) | detected**) |
| 10 | 46 | 64 |
| 1 | 473 | 662 |
| 0.1 | 4,745 | 6,639 |
| 0.05 | 9,496 | 13,288 |

*Example for upper bound: an upper bound of 10% means that there is 95% (or 99%) confidence that the true fraction of GM plants in the population is less than 10%.
**Infinite population assumed, binomial distribution used. Sample size less than 10% of population size and population size is greater than 5000. If the sample size/population size is at least 10% and/or the population size is 5000 or less, then the distribution used will be hypergeometric.

Binomial distribution is a frequency distribution of the possible number of successful outcomes in a given number of trials in each of which there is the same probability of success.

### III. EXAMPLE

The following illustrates the methods of the present invention. The example is given solely for the purpose of illustration, and is not to be construed as limiting the present invention since many variations thereof are possible without departing from its spirit and scope.

3 circular fields for potatoes (areas of 125, 135, and 135 acres each for a total of 395 acres) are planted from one seed source and the variety is Russet Burbank. These 3 fields make up a single population. (In this example, the population is chosen based on the fact that seeds planted in all 3 fields are obtained from the same seed supplier.) Before harvest and plant defoliation, 4603 plant leaf samples (distinct plants) are taken from the fields in the following manner:

GM Potato Sampling
Input Field dimensions

| | | | Distance | |
|---|---|---|---|---|
| | Pivot Length | | between | |
| Pivot # | (if applicable) | # of Towers | Towers | Field Area |
| 1 | 1315 ft | 8 | 164 ft | 125 Acres |
| 2 | 1370 ft | 9 | 152 ft | 135 Acres |
| 3 | 1370 ft | 9 | 152 ft | 135 Acres |

The term “pivot” refers to a delivery arm (i.e., in a circular field) for delivering, via spraying, water or water-soluble additives to the field. The length of the pivot is the radius of the circular field.

The term “tower” refers to a support wheel for the pivot and the tower's passage through the field creates a track, which becomes a natural path for walking through the field. Depending on field size, most pivots include multiple towers. Starting from the first tower, these tracks are of increasing length (circumference) until the final one equals the circumference of the field.

The following provides the field sample methodology:

GM Potato Sampling
Calculation of Distance Between Sampling Points

| | # of | Distance | | | | | | | | |
|---|---|---|---|---|---|---|---|---|---|---|
| | Samples/ | between | | | | | | | | |
| Pivot # | Field | Samples | Towers Sampled | | | | | | | |
| 1 | 1451 | 14 ft | 2 | | 4 | | 6 | | 8 | |
| 2 | 1576 | 15 ft | | 3 | | 5 | | 7 | | 9 |
| 3 | 1576 | 15 ft | | 3 | | 5 | | 7 | | 9 |

↑ ↑ ↑ ↑ ↑
Sampling Methodology

| | | | |
|---|---|---|---|
| Total Field Area: | 395 Acres | Total Samples: | 4603 |

The sampling in this case is along the pivot towers (tracks that support the pivot), but sampling could occur in a grid pattern, or any other pattern that provides a representative cross section of the population. For field 1, the sampling occurs along the towers 2, 4, 6, and 8 every 14 feet, while for fields 2 and 3, the sampling is along towers 3, 5, 7, and 9 every 15 feet.

Leaf samples from plants are collected and are grouped into 9 bunches of about 500 leaves each (from distinct plants), and sent to Genescan USA Inc. (Belle Chasse, La.) for analysis. The number of plants combined is dependent on the detection limit for the GM analysis. In this example, the level of quantification (LOQ) is 0.1%, meaning that one could detect and quantify one in 1000 plants (leaves) that are genetically modified. By applying a 2:1 safety margin (due to sample non-uniformity), the number of samples analyzed simultaneously could be dropped to about 500.

Genescan analyses of the samples are performed according to the following procedure. (It will be recognized that this example relates to one embodiment concerning the testing of potato plants where the plant products are eventually exported to Japan. Accordingly, the statistical analysis, permitted level of GM content, etc. are dictated by the regulations currently set forth by the relevant governmental agencies. Of course, the parameters used could change depending on where and how the plant products are eventually distributed. As such, this example should not be interpreted to limit in any way the scope of the invention as claimed.)

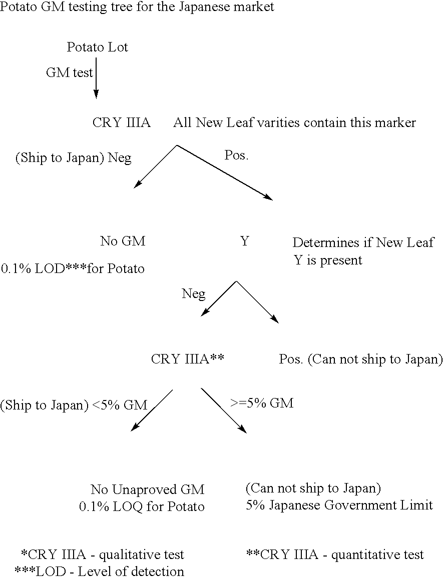

Here, the potato lot represents the 3 fields from the same seed supplier (presented above). The first test classifies the material as ‘New Leaf containing’ or ‘New Leaf Free.’ If all the samples are New Leaf Free, product from those 3 fields can be used for the Japanese production. If the sample does contain New Leaf GM, then a second test is performed that determines the presence or absence of New Leaf Y GM. If New Leaf Y is present in any of the samples, product cannot be shipped to Japan. If, on the other hand, the Sample contains no New Leaf Y, but contains some other New Leaf modification, then a quantitative test for GM content in the sample is obtained. If the total amount of adventitious New Leaf GM contamination is less than 5%, then the product from those fields can again be shipped to Japan.

Based on the foregoing, the testing results from the genetic testing facility are used to determine whether the plant products from the population tested are “non-GM” (as established in view of the relevant Japanese standards, which are applicable to this population of plants). Because the results from the plant samples tested indicate the products are non-GM, the plant products of the population can be combined with other non-GM plant products for further handling/processing. The plant products would not be combined with products that are determined via the present methods to be GM plant products, thereby avoiding unwanted contamination of non-GM products.

### INCORPORATION BY REFERENCE

All of the aforementioned patents, patent applications, publications, and other references are herein incorporated by reference in their entirety.

What is claimed is:

1. A method for determining the relative number of genetically modified plant products in a population of plants so as to characterize the plant products as being either ‘genetically modified’ or ‘non-genetically modified’, the method comprising genetic testing of samples obtained from a representative number of the plants of the population.

2. The method of claim 1 wherein either (i) the plants do not require pollination to reproduce or (ii) the plant product is other than the plant seed; the method comprising obtaining samples from the group consisting of the plant stem, plant leaves, plant flowers, plant roots, the plant product, and any combination thereof.

3. The method of claim 1 wherein the plants require pollination to reproduce and the plant product is the seed of the plant; the method comprising obtaining samples from the plant product.

4. The method of claim 1 wherein the plants produce a product selected from the group consisting of fruits, vegetables, grains, tubers, cereals, and legumes.

5. The method of claim 4 wherein the plants produce potatoes.

6. The method of claim 1 wherein the genetic testing comprises the use of an enzyme linked immunosorbent assay (ELISA), a polymerase chain reaction (PCR) assay, or a combination of the two assays.

7. The method of claim 1 wherein the genetic testing is conducted prior to harvesting the plant products of the population of plants.

8. The method of claim 1 wherein the genetic testing is conducted after harvesting of the plant products of the population but prior to combining plant products of the population with plant products from one or more other populations.

9. The method of claim 1 wherein samples are taken from a sufficient number of plants of the population to determine whether the GM content of the population is less than a predetermined level at at least a 90% confidence interval.

10. The method of claim 1 wherein samples are taken from a sufficient number of plants of the population to determine whether the GM content of the population is less than a predetermined level at at least a 95% confidence interval.

11. The method of claim 10 wherein the predetermined level is not more than about 5%.

12. The method of claim 1 wherein samples are taken from a sufficient number of plants of the population to determine whether the GM content of the population is less than a predetermined level at at least a 99% confidence interval.

13. A method for reducing the level of contamination of non-genetically modified plant products with genetically modified plant products, the method comprising the steps of:
a. utilizing data obtained from genetic testing of samples obtained from plants from a plurality of populations of plants prior to combining plant products from the different populations; and
b. determining whether plants from any of the populations are genetically modified.

14. The method of claim 13 wherein either (i) the plants do not require pollination to reproduce or (ii) the plant product is other than the plant seed; wherein the data utilized in step (a) results from genetic testing of samples from the group consisting of the plant stem, plant leaves, plant flowers, plant roots, the plant product, and any combination thereof.

15. The method of claim 14 wherein the plants produce a product selected from the group consisting of fruits, vegetables, grains, tubers, cereals, and legumes.

16. The method of claim 15 wherein the plants produce potatoes.

17. The method of claim 13 wherein the genetic testing comprises the use of an enzyme linked immunosorbent assay (ELISA), a polymerase chain reaction (PCR) assay, or a combination of the two assays.

18. The method of claim 13 wherein samples are taken from a sufficient number of plants of the population to determine whether the GM content of the population is less than a predetermined level at at least a 95% confidence interval.

19. The method of claim 18 wherein the predetermined level is not more than about 5%.

20. The method of claim 19 wherein the predetermined level is not more than about 1%.

21. The method of claim 13 wherein samples are taken from a sufficient number of plants of the population to determine whether the GM content of the population is less than a predetermined level at at least a 99% confidence interval.

22. A method for reducing the level of contamination of non-genetically modified plant products with genetically modified plant products, the method comprising the steps of:
a. utilizing data obtained from genetic testing of samples obtained from plants from a plurality of populations of plants prior to combining plant products from the different populations;
b. determining whether plants from any of the populations are genetically modified; and
c. combining only plant products from populations that are determined not to be genetically modified.

23. The method of claim 22 further comprising the step of determining the relative level of contamination in a population that will result in that population being characterized as genetically modified.
